(19) United States
(12) Patent Application Publication
(10) Pub. No.: US 2024/0247032 A1
(43) Pub. Date: Jul. 25, 2024

(54) METHOD OF SYNTHESIZING DENDRIMERIC AMPHIPHILE

(71) Applicant: KAOHSIUNG MEDICAL UNIVERSITY, Kaohsiung City (TW)

(72) Inventors: Chai-Lin Kao, Kaohsiung City (TW); Hui-Ting Chen, Kaohsiung City (TW); Vijayasimha Molakaseema, Andrapradesh (IN); Anand Selvaraj, Tamil Nadu (IN)

(73) Assignee: KAOHSIUNG MEDICAL UNIVERSITY, Kaohsiung City, TW (TW)

(21) Appl. No.: 18/558,364

(22) PCT Filed: Aug. 23, 2021
(86) PCT No.: PCT/CN2021/114031

§ 371 Date: Oct. 31, 2023

(51) Int. Cl. C07K 14/00; A61K 47/34; C08G 83/00

(52) U.S. Cl. CPC C07K 14/001; A61K 47/34; C08G 83/003

(57) ABSTRACT

A method for preparing amphiphiles with dendrimeric skeletons by a solid phase linker approach.

### FIELD OF THE INVENTION

The present invention relates a preparing method of amphiphiles with dendrimeric skeletons by a solid phase approach.

### BACKGROUND OF THE INVENTION

The amphiphiles with dendrimeric skeletons (shorten named AD) equipped with hydrophilic and hydrophobic characteristics provide a subclass of molecules. Their self-assembling nature makes them excellent carriers for various compounds such as drugs, imaging agents, and genes. Besides, it also benefits the artificial vaccine and the antifouling material. In a classical divergent liquid phase method, molecular construction usually requires multi-reaction steps and a massive number of reactions. Meanwhile, it is convenient to prepare a compound through a convergent approach, in which the hydrophobic and hydrophilic components are designed individually and coupled at the final step. However, the incompatible properties of the hydrophobic and hydrophilic segments limit available solvents. Moreover, the slow coupling reaction between two large molecules further hampers their synthetic efficiency and usually consumes many compounds. Moreover, both synthetic approaches in liquid-phase experience incomplete reactions and a more extended preparation period, majorly due to complicated purification. The by-products with structural similarity are usually given in a product mixture and cause difficulty isolating the desired product. Meanwhile, amphiphilic nature causes difficulty in considering solubility, which limits available solvents in preparation and purification. This fact further damages the feasibility of liquid-phase preparation of AD. Moreover, the purity of the final compounds does not apply to the clinical applications. Until now, few solid-phase strategies are applied to prepare AD.

### SUMMARY OF THE INVENTION

The present invention provides a solid-phase approach for the synthesis of amphiphiles with dendrimeric skeletons, in which peripheral groups could be easily decorated with a wide range of functionalities. Remarkably, the hydrophobic moiety was introduced at the cleavage step. This fact improves the preparative efficiency and products' purity. At the cleavage step, the cocktail solution is necessary to prevent hydrolytic side-product and offer desired compounds by improving hydrophobic segments' solubility. Besides, the necessity of applying microwave has been identified to enhance the yield and shorten the reaction time. In short, this invention provides a convenient and economical approach to prepare broad-spectra of conjugates for multi-purposed applications.

### BRIEF DESCRIPTION OF THE DRAWINGS

FIG. 1 shows the overview of the synthetic approaches of the amphiphiles with dendrimeric skeletons in this present invention.

FIG. 2 shows the synthetic approach of the compound of formula (Ib). R is H, side chain of amino acid or protected side chain of amino acid. PG: protecting group.

FIG. 3 shows the approach for anchoring of the compound of formula (Ib) on the resin. M is a solid support material; X is an amino carboxylate; and Y is a protecting group to protect the N-terminal of X.

FIG. 4 shows the synthetic procedures for generation-1 (G₁) lysine core. M is a solid support material.

FIG. 5 shows the synthetic procedures for generation-2 (G₂) lysine core. M is a solid support material.

FIG. 6 shows the synthetic procedures for generation-3 (G₃) lysine core. M is a solid support material.

FIG. 7 shows the synthetic procedures for generation-4 (G₄) lysine core. M is a solid support material.

FIG. 8 shows the synthetic procedures for hybrid generation-1 (G₁) dendrimer. M is a solid support material.

FIG. 9 shows the synthetic procedures for hybrid generation-2 (G₂) dendrimer. M is a solid support material.

FIG. 10 shows on-bead activation for resin-bound Dbz peptide-dendron. M is a solid support material.

FIG. 11 shows nucleophilic substitution to de-attach compounds from a solid support. FIG. 11(a) shows that the compound with mono amino group is used as the nucleophile. FIG. 11(b) shows that the compound with multi amino groups is used as the nucleophile. FIG. 11(c) shows that synthetic dendron or dendrimer is used as the nucleophile.

### DETAILED DESCRIPTION OF THE INVENTION

The term “a” or “an” as used herein describes elements and ingredients of the present invention. This term is only for the convenience of description and the basic idea of the present invention. The description should be understood as comprising one or at least one, and unless otherwise indicated by the context, singular terms include pluralities and plural terms, including the singular. When used in conjunction with the word “comprising” in a claim, the term “a” or “an” may mean one or more than one. The term “surface” as used herein describes the terminal groups of dendrimers or dendrons.

The term “amphiphiles” as used herein describes a compound which contains both hydrophobic and hydrophilic residues. They can be prepared by attacking a hydrophilic residue with a hydrophobic residue; or oppositely, by attacking hydrophobic residue with a hydrophobic residue.

The present invention provides a method for synthesizing amphiphilic dendrimers, comprising: (a) reacting a compound of formula (IIb) with at least one diamino carboxylate having two protecting groups for replacing Y on the compound of formula (IIb) to develop branched skeletons and modifying the terminal groups of the branched skeleton with functional moieties to obtain a compound of formula (IIIb),

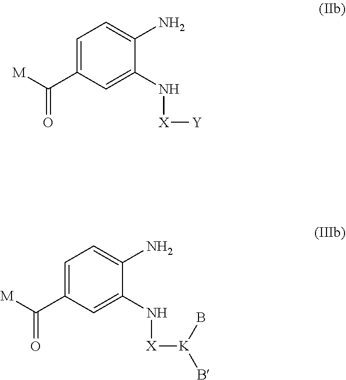

wherein M is a solid support material;
X is an amino carboxylate;
Y is a protection group to protect the N-terminal of X;
K is a moiety with branched diamine;
B and B′ are K₍₂_{ⁿ}₋₁)Z₂_{ⁿ}, wherein n is 0 to 5 and Z is a functional moiety comprising H, amino acids, peptides, acids, sugars, targeting ligands, imaging molecules, therapeutic agents, or peptidomimetic molecules;
provided that B is not the same as B′, at least one of Z, K or n of B is different from B′;
(b) reacting the compound of formula (IIIb) with nitrite in a polar solvent to obtain a compound of formula (IVb) having a benzotriazole structure,

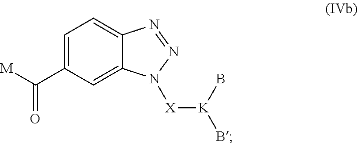

and
(c) reacting the compound of formula (IVb) with one amine having hydrophobic residues in a mixed solution containing a hydrophilic solvent and a hydrophobic solvent to obtain a compound of formula (Vb),

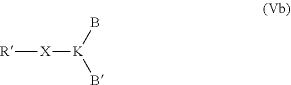

wherein R′ comprises amino groups having hydrophobic residues; or reacting at least two compounds of formula (IVb) with corresponding di- or multi-amine having hydrophobic residues in a mixed solution containing a hydrophilic solvent and a hydrophobic solvent to obtain a compound of formula (Vb′),

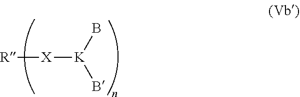

wherein R″ comprises branched di- or multi-amino amino groups having hydrophobic residues, and n>1.

In one embodiment, the above method further comprises step (pre-a3), before the step (a), comprising reacting a compound of formula (IIa) with a compound having X-Y to obtain the compound of formula (IIb).

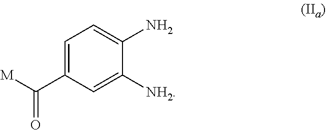

In another embodiment, the above method further comprises step (pre-a2), before step (a), comprising immobilizing a compound of formula (Ib) on a solid support material to obtain the compound of formula (IIb),

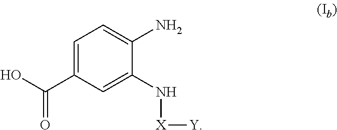

In a preferred embodiment, the above method further comprises step (pre-a1), before step (pre-a2), comprising reacting a compound of formula (Ia) with a compound having the X-Y to obtain the compound of formula (Ib),

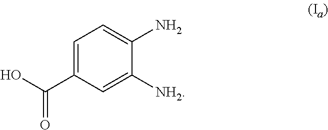 [SMILES: Nc1ccc(C(=O)O)cc1N]

In one embodiment, the at least one diamino carboxylate is not limited but to aliphatic diamino carboxylate, cyclic diamino carboxylate, aromatic diamino carboxylate or heterocyclic diamino carboxylate. In a preferred embodiment, the aliphatic diamino carboxylates comprises lysine, ornithine, homolysine, 2,7-diaminoheptanoic acid, 5-amino-2-amino-pentanoic acid, 3-(bis(3-aminopropyl)amino)propanoic acid or polyethylene glycol.

In another embodiment, the at least one diamino carboxylate is amino acids and/or polyethylene glycol. In a preferred embodiment, the at least one diamino carboxylate comprises Fmoc-Lys(Fmoc)-OH, Boc-Lys(Boc)-OH, Fmoc-Lys(Boc)-OH and Fmoc-Lys(Alloc)-OH), Fmoc-Lys(Dabcy)-OH, Fmoc-Lys(Caproyl)-OH, Fmoc-Lys(Z)-OH, Fmoc-Lys(Crotonyl)-OH, Fmoc-Lys(Mmt)-OH, Fmoc-Lys(Dansyl)-OH, Fmoc-Lys(ivDde)-OH, Fmoc-Lys(Teoc)-OH, Fmoc-Lys(Mtt)-OH, or Fmoc-Lys(2-CIZ)-OH.

In another embodiment, the solid support material comprises controlled-pore glass, magnetic beads, Rink amide resin, Tentagel resin, Wang resin, Merrifield resin, MBHA resin, PAM resin, PAL resin, Sieber Amide resin, trityl resin, chlorotrityl resin, Polyethylene Glycol-Polystyrene resin, Weinreb resin, oxime resin, DHP resin or Safety-catch resin.

As used herein, the X comprises natural, unnatural and unusual amino acids. The term “natural amino acids” comprises the natural occurring form, i.e., the L form (except for glycine) of glycine (Gly), alanine (Ala), valine (Val), leucine (Leu), isoleucine (Ile), serine (Ser), threonine (Thr), phenylalanine (Phe), tyrosine (Tyr), tryptophan (Trp), cysteine (Cys), methionine (Met), proline (Pro), hydroxyproline, aspartic acid (Asp), asparagine (Asn), glutamine (GIn), glutamic acid (Glu), histidine (His), arginine (Arg), and lysine (Lys). The term “unnatural amino acids” comprises all natural amino acids defined as above, in their D form and the term “unusual amino acids” comprises citrulline (Cit), hydroxyproline (Hyp), norleucine (Nle), 3-nitrotyrosine, nitroarginine, ornithine (Orn), naphtylalanine (Nal), methionine sulfoxide or methionine sulfone.

In another embodiment, the X comprises:

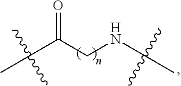

wherein n is an integer, from 1 to 10; or

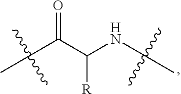

wherein R is H, —CH₃, —CH(CH₃)₂, —CH₂CH(CH₃)₂, —CH(CH₃)CH₂CH₃. CH₂C₆H₅; the O-PG derivatives of —CH₂COOH, —CH₂CH₂COOH, —CH₂OH, —CH₂CH(CH₃)OH or —CH₂C₆H₄OH; the S-PG derivatives of CH₂SH or —CH₂CH₂SCH₃; the N-PG derivatives of —CH₂CH₂CONH₂, —CH₂CONH₂, —CH₂CH₂CH₂CH₂NH₂ or —CH₂CH₂CH₂NHC(NH)NH₂; or the N-PG derivatives of

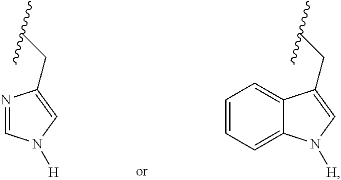

wherein PG is a protecting group to protect N, O, or S. Besides, the protecting group (PG) can be removed in prior to obtain final compound.

In one embodiment, the Y comprises the protecting groups of N-terminal of coupled natural, unnatural and unusual amino acids. In a preferred embodiment, the Y comprises 9-fluorenylmethoxycarbonyl (Fmoc), tert-butoxycarbonyl (Boc), benzyl carbamates (Cbz), benzy (Z) or alloxycarbonyl (Alloc) groups.

In another embodiment, the K is the moiety which derived from diamino carboxylates having same or orthogonal protecting groups. Therefore, the step (a) in the method comprises two reactions as follows: (1) B=B′: The compound of formula (IIb) coupled to a first K having same protecting groups on amines, later the protecting group on amines are removed, then the amino group of the first K coupled to the second K having same protecting groups. In particular, this reaction must uses the K having same protecting groups during the these above steps. Repeated deprotection and coupling steps to develop a desired skeleton on the X of the compound of formula (IIb). Later modifying the surface of the branched skeleton with functional moieties to obtain the compound of formula (IIIb); and (2) B≠B′: The compound of formula (IIb) coupled to a first K having orthogonal or same protecting groups on amines, later the protecting group on amines are removed, then the amino group of the first K coupled to a second K having orthogonal or same protecting groups. Therefore, the protecting groups can be same or different useded in this reaction. In particular, this reaction must uses at least one K having orthogonal protecting groups during the these above steps. Repeated deprotection and coupling steps to develop a desired skeleton on the X of the compound of formula (IIb). Later modifying the surface of the branched skeleton with functional moieties to obtain the compound of formula (IIIb).

In one embodiment, the B or B′ is a Z moiety that derived from the compounds comprising amino acids, peptides, (including cyclic and branched peptides), acids (including alkyl and aromatic acids), sugars, fluorescence molecules, imaging agents, targeting ligands, therapeutic agents, or peptidomimetic molecules.

The targeting ligands that exhibit high affinity for pathologic cells of given tissues. It enables to recognize the specific antigens or receptors on target cells. In another embodiment, the targeting ligands comprise organic molecule, carbohydrates, monoclonal antibodies, peptides, proteins, vitamins, and aptamers.

In one embodiment, the form of the B or B′ is the branched skeletons comprising KZ₂, K(KZ₂)₂, K(K(KZ₂)₂)₂, K(K(K(KZ₂)₂)₂)₂, or K(K(K(K(KZ₂)₂)₂)₂)₂. Therefore, the branched skeletons form dendrimers or dendrons and the functional moieties Z is used for modifying with the surfaces of the dendrimers or dendrons. The branched skeletons were obtained from stepwise synthesis based on the above method.

In another embodiment, the detail description for two reactions in the step (c) is recited as follows: (1) the attack strategy of the monoamine: attacking the X on the compound of formula (IVb) and replacing its benzotriazole moiety with an amine having hydrophobic residues. Therefore, the compound of formula (Vb) having chemical structure R′-XKBB′ is obtained; and (2) the attack strategy of the di- or multi-amine: attacking the X and replacing benzotriazole moiety on at least two compounds of formula (IVb) with corresponding at least one amino group on di- or multi-amine having hydrophobic residues. Therefore, the compound of formula (Vb′) having chemical structure R″-(XKBB′)ₙ (n is an integer and >1) is obtained.

According to the present invention, the R′ is amino groups having hydrophobic residue. In one embodiment, the hydrophobic residue is included in single or/and double aliphatic compounds, arenes, and cyclic peptides. In another embodiment, the hydrophobic residue of R′ comprises a group serving for a host-guest interaction, wherein the R′ comprises adamantane. In another embodiment, the hydrophobic residue of R′ is an aggregative, wherein the aggregative comprises pyrene. In one embodiment, the hydrophobic residue of R′ is a metal binder, wherein the metal binder comprises dopamine, pyrraolze, imidazoles, pyridines, bipyridines, urea, thiourea or other ligands.

In another embodiment, the R′ comprises:

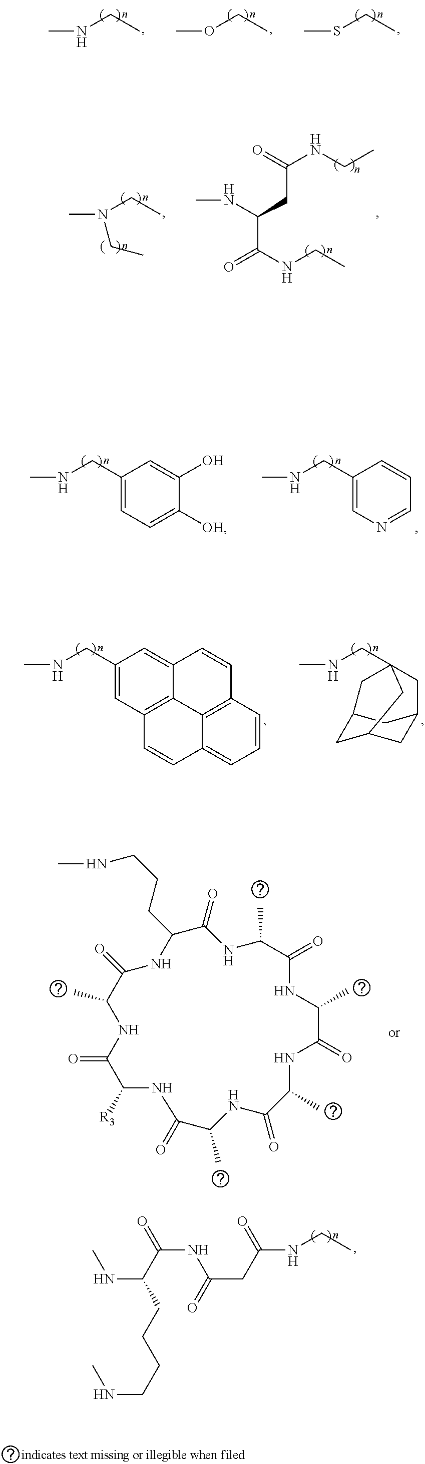

wherein n=0 to 20, and R₃ is a side chain of amino acids.

In one embodiment, the R″ comprises a diamino or multi-amino moieties on hydrophobic skeleton. In a preferred embodiment, the form of the skeleton comprises aliphatic skeleton, cyclic skeleton, aromatic skeleton or heterocyclic skeleton. In a more preferred embodiment, those skeletons are derived from the compounds comprising dendrimer, diaminoalkanes, diaminocycloalkane, diaminobenzene, or aminomethyl piperidine.

In one embodiment, the nitrite comprises sodium nitrite, potassium nitrite, ethyl nitrite, butyl nitrite, t-butyl nitrite, isobutyl nitrite, pentyl nitrite, nitrite, iso-amyl nitrite, dicyclohexylamine nitrite, or isopentyl tetrabutylammonium nitrite.

In one embodiment, a solvent is used in the reaction of the step (a), wherein the solvent comprises dichloromethane (DCM), N,N-dimethylacetamide (DMAC) or N,N-dimethylformamide (DMF).

In another embodiment, the polar solvent in the step (b) comprises DCM, DMAC, DMF, N-methyl-pyrrolidone (NMP), dichloroethane, trichloroethane or water.

In another embodiment, the hydrophilic solvent in the step (c) comprises a polar solvent. In a preferred embodiment, the polar solvent comprises dimethyl sulfoxide (DMSO), N,N-dimethylformamide (DMF), N,N-dimethylacetamide (DMAC), N-methyl-pyrrolidone (NMP) or N-butylpyrrolidinone (NBP). In one more preferred embodiment, the polar solvent comprises NMP or NBP.

In one embodiment, the hydrophobic solvent in the step (c) comprises a less polar solvent including dichloromethane (DCM), dichloroethane (DCE), dichlorobenzene (DCB), 1,1,2-trichloroethane (TCE), tetrahydrofurane (THF), dioxane or ethyl acetate.

In another embodiment, the reacting temperature of the mixed solution ranges from 1 to 150° C. In a preferred embodiment, the reacting temperature of the mixed solution ranges from 10 to 100° C. In a more preferred embodiment, the reacting temperature of the mixed solution ranges from 20 to 65° C.

In one embodiment, the volume ratio of the hydrophilic solvent and the hydrophobic solvent ranges from 50:0 to 1:15. In a preferred embodiment, the volume ratio of the hydrophilic solvent and the hydrophobic solvent ranges from 25:0 to 1:10. In a more preferred embodiment, the volume ratio of the hydrophilic solvent and the hydrophobic solvent ranges from 10:0 to 1:5.

In another embodiment, the reaction of the mixed solution is in a microwave environment.

In one embodiment, the microwave power of the microwave environment ranges from 10 to 250 W. In a preferred embodiment, the microwave power of the microwave environment ranges from 50 to 200 W. In a more preferred embodiment, the microwave power of the microwave environment ranges from 80 to 150 W.

In another embodiment, the temperature of the microwave environment ranges from 1 to 150° ° C. In a preferred embodiment, the temperature of the microwave environment ranges from 25 to 100° C. In a more preferred embodiment, the temperature of the microwave environment ranges from 40 to 75° C.

In one embodiment, the reaction time in the microwave environment ranges 5 to 40 min. In a preferred embodiment, the reaction time in the microwave environment ranges from 6 to 25 min. In a more preferred embodiment, the reaction time in the microwave environment ranges from 8 to 15 min.

In the step (c) of the above method, the compound of formula (Vb) or (Vb′) is obtained by nucleophilic substitution of the benzotriazole on the compound of formula (IVb) or (IVb′) with a nucleophilic moiety such as amino group. When it is necessary, the protecting groups on the compound of formula (Vb) or (Vb′) will be removed under typical protocols according to the type of protecting groups. In another embodiment, the method further comprises a step (d), after the step (c), comprising removing the protecting groups of the compound of formula (Vb) or (Vb′).

The amphiphilic dendrimers are widely used as the building block of supra-molecules by the assembly process. The structures of the amphiphilic dendrimers are leading to the various architecture of supra-molecules. Therefore, the assembling supra-molecules possess programmable cavities, surface functionalities. The resulting assembling supra-molecules are widely used as delivery vehicles for different freight molecules, such as drugs, genes, and imaging agents. Moreover, the diverse assembling supra-molecules can be produced by mixing several amphiphilic dendrimers. Therefore, an assembly body with various functions, such as targeting delivery, and multi-modalities, can be prepared.

Besides, the amphiphilic dendrimers are also well-known for various purposes, including enhancing vaccines' immune response and antifouling effect. Moreover, the amphiphilic dendrimers can form lyotropic liquid crystals, which is vital in the current industry.

### EXAMPLES

The examples below are non-limiting and are merely representative of various aspects and features of the present invention.

The scheme of the preparing method of the amphiphiles with dendrimeric skeletons

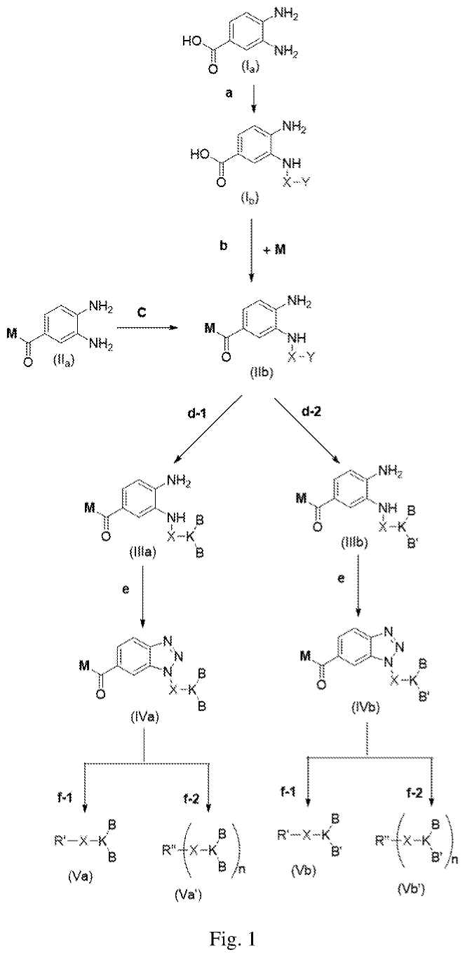

FIG. 1 showed the scheme for preparing the amphiphiles with dendrimeric skeletons of the present invention. In FIG. 1, M in the compounds was a solid support material. Here, M represented solid support resins like chlorotrityl chloride (CTC) resin, Rink Amide resin, Wang resin, Merrifield resin etc. for the dendritic core synthesis. X in the compounds was an amino carboxylate. In particular, X represented the amino carboxylates formed from reacting the compound of formula (Ia) or (IIa) with protected natural, unnatural and unusual amino acids. The term “natural amino acid” meant the natural occurring form, i.e., the L form (except for glycine) of glycine (Gly), alanine (Ala), valine (Val), leucine (Leu), isoleucine (Ile), serine (Ser), threonine (Thr), phenylalanine (Phe), tyrosine (Tyr), tryptophan (Trp), cysteine (Cys), methionine (Met), proline (Pro), aspartic acid (Asp), asparagine (Asn), glutamine (Gln), glutamic acid (Glu), histidine (His), arginine (Arg), and lysine (Lys). The term “unnatural amino acids” comprised all natural amino acids defined as above in their D form and the term “unusual amino acids” i.e. citrulline (Cit), hydroxyproline (Hyp), norleucine (Nle), 3-nitrotyrosine, nitroarginine, ornithine (Orn), naphtylalanine (Nal), methionine sulfoxide or methionine sulfone. Y in the compounds was a protecting group to protect N-terminal of X. In particular, Y represented the protecting groups of N-terminal of coupled natural, unnatural and unusual amino acids. For example, Y comprised 9-fluorenylmethoxycarbonyl (Fmoc), tert-butoxycarbonyl (Boc), and benzyl carbamates (Cbz) groups. K in the compound was a moiety obtained from reacting branched diamine with the compound of formula (IIb) removed Y. In particular, K represented the branched diamine core for the synthesis of dendritic core. The branched diamine comprised lysine amino acids, 3-(bis(3-aminopropyl)amino)propanoic acid, polyethylene glycol and also comprised the branched diamine core with different protection groups with specific deprotection condition (orthogonality) for the synthesis of two different halves for hybrid dendron. For example, the branched diamine comprised Fmoc-Lys(ivDde)-OH, Fmoc-Lys(Boc)-OH and Fmoc-Lys(Alloc)-OH). B or B′ in the compounds was K₍₂_{ⁿ}₋₁₎Z₂_{ⁿ}(n=0 to 5) and Z was a functional moiety decorated on B or B′ with the compounds comprising H, amino acids, peptides, acids, sugars, targeting ligands, imaging molecules, therapeutic agents, or peptidomimetic molecules. B′ was a different half other than B moieties, and at least one Z, K or n different from B. R′ was various amino groups having hydrophobic residues such as single or double alkyl chains with different chain lengths by reacted the compound of formula (IVa) or (IVb) with aminoalkanes, cyclic peptides, host-guests (adamantyl), aggregates (pyrene), metal binders (dopamine), hydrophobic dyes or imaging agents. For example, R′ comprised:

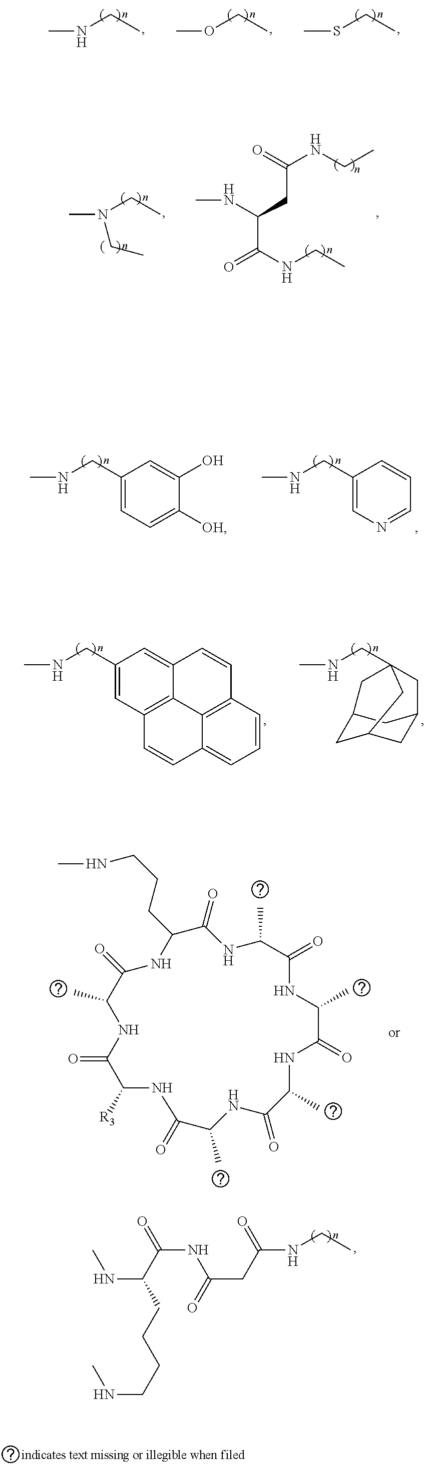

wherein n=0 to 20, and R₃ is a side chain of amino acids. R″ comprised various di- or multi-amino groups having hydrophobic residues by reacted the compound of formula (IVa) or (IVb) with aliphatic, cyclic, aromatic or heterocyclic, or an amphiphilic dendrimer skeletons installed branched di- or multi-amines.
   Synthetic Method of the Amphiphiles with Dendrimeric Skeletons

Abbreviations: DMAC: N,N-dimethylacetamide; DMF: N,N-dimethylformamide; DCM: dichloromethane; DMSO: dimethyl sulfoxide; NMM: N-methylmorpholine; NMP: N-methyl-pyrrolidone; NBP: N-butylpyrrolidinone; DCE: dichloroethane; DCB: dichlorobenzene; TCE: 1,1,2-trichloroethane; THF: tetrahydrofurane; TEA: triethylamine; DIPEA: N,N-diisopropylethylamine; DBU: 1,8-diazabicyclo[5.4.0]undec-7-ene; DEA: diethylamine; Dbz: 3,4-diaminobenzoic acid; PG: protecting group; HATU: (1-[Bis(dimethylamino)methylene]-1H-1,2,3-triazolo[4,5-b]pyridinium 3-oxide hexafluorophosphate; HBTU: hexafluorophosphate benzotriazole tetramethyl uronium; HCTU: O-(1H-6-chlorobenzotriazole-1-yl)-1,1,3,3-tetramethyluronium hexafluorophosphate; TFA: trifluoroacetic acid; ACN: acetonitrile; ivDde: 1-(4,4-dimethyl-2,6-dioxocyclohex-1-ylidene)-3-benzotriazol-1-methylbutyl; Boc: tert-butyloxycarbonyl; PyBop: yloxytripyrrolidinophosphonium hexafluorophosphate; Alloc: allyloxycarbonyl.

### Reagents

1. Fmoc de-protection solution: 20% piperidine (v/v) in DMF.

2. Hydrazin solution: 2% of N₂H₄·H₂O (purity 98%) in DMF.

3. Activation reagent: 5% of NMM (v/v) in DMF.

4. Coupling reagent: HBTU (3.5 eq.) for single amide bond.

5. Cleavage reagent: 95% of TFA (v/v) in deionized water.

### Synthetic Process for the Synthesis of Self-Assemble Supramolecules

According to the synthetic approach in FIG. 1, solid phase synthesis of self-assemble molecules (the amphiphiles with dendrimeric skeletons) started with the synthesis of PG-amino acid-Dbz-OH (step (a)) moiety which was anchored to the solid support (step (b)) to obtain the compound of formula (IIb). Alternatively, a common amino acid coupling after the compound of formula (IIa) formation were applied to obtain the compound of formula (IIb) (step (c)). Further, dendritic moiety was constructed with repeating solid phase strategies to reach desired generation number through iterative N-protection group removal and coupling with diamino carboxylates. In step (d-1), the construction of regular dendrons were prepared by repeating using carboxylates with same N-protected groups on their diamino groups. In step (d-2), at least one carboxylate with different N-protected groups on diamino groups was used and selectively deprotected one-by-one to construct hybrid dendrons.

After dendron construction, on-bead Dbz (O-aminoanilide) moiety was activated to form resin bound acylated benzotriazole with alkyl nitrite (step (e)). After on-bead activation, resin was washed thoroughly with DMF and DCM further followed for the nucleophilic substitution. In step (f-1), the benzotriazole linker on the compound of formula (IVa) or (IVb) was substituted with a nucleophile with hydrophobic residue to obtain nucleophile-X conjugate and detach the hydrophilic residue from resin. Finally, an amphiphilic compound was obtained. Alternatively, a molecule with two or more nucleophilic functionalities was used to attack two compounds of formula (IVa) or (IVb) and replaced benzotriazole linker to obtain X-nucleophile-X conjugate (step (f-2)). The selection of solvent system for the nucleophile substitution depends on the nature of the nucleophiles. For example, different DCM and DMF cocktail combination is needed for C18 hydrophobic alkane and pyrene; other solvent combination or sole solvent was used for the other hydrophilic groups like adamantly and dopamine moiety.

Finally, nucleophilic substitution deattached the compound from resin to present it in a solution. Removal of solvent gets crude protected compound. Deprotection condition varies on each protection group used. Purification was followed based on the nature of the compound.

The detail description of the steps of the synthetic method was as follows:

### Step (a): General Synthetic Procedure for Protection Group-Amino Acid-Diaminobenzoic Acid (PG-Amino Acid-Dbz-OH) (the Compound of Formula (Ib))

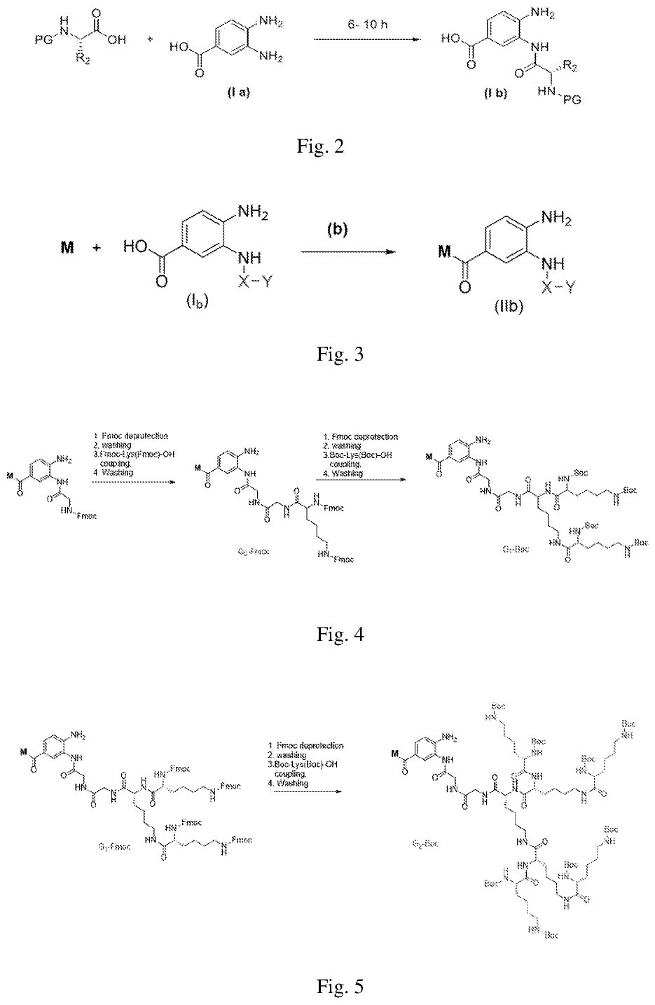

As shown in FIG. 2, to the solution of 3,4-diaminobenzoic acid (the compound of formula (Ia)) (1.2 eq.) in DMF was added a solution of PG-amino acid-OH (1 eq.). The reaction was activated with coupling reagent (such as HBTU, HCTU, HATU, PyBop) (1 eq.) and base (TEA, DIPEA, NMM, DBU) (2 eq.) in DMF for 2-10 min. The resulting mixture was stirred at room temperature for 6-10 hr. After removal of solvent, the resulting mixture was dissolved in water and added hexane until precipitate appear. The resulting mixture was stirred for additional 1-8 hr to get free flow solid, which was filtered and dried. The solid was purified by flash column chromatography with different combinations of dichloromethane and MeOH based on the solubility to get the product with good purity and yields (60-85%).

Based on the above procedure, various PG-amino acid-Dbz-OH (the compound of formula (Ib)) were synthesized by various reaction conditions according to the species of PG-amino acid-OH. The conditions were listed in the table 1.

TABLE 1
The conditions for synthesis of the compound of formula (Ib)

| | Reactant | Coupling | | Reaction | Yield |
|---|---|---|---|---|---|
| Entry | (PG-amino acid-OH) | reagent | Base | time (h) | (%) |
| 1 | Fmoc-Ala-OH | HBTU | DIPEA | 6 | 80 |
| 2 | Fmoc-Cys(Trt)-OH | HBTU | DIPEA | 9 | 65 |
| 3 | Fmoc-Asp(OtBu)-OH | HBTU | DIPEA | 8 | 85 |
| 4 | Fmoc-Glu(OtBu)-OH | HBTU | DIPEA | 10 | 80 |
| 5 | Fmoc-Phe-OH | HBTU | DIPEA | 9 | 75 |
| 6 | Fmoc-Gly-OH | HBTU | DIPEA | 6 | 86 |
| 7 | Fmoc-His (Trt)-OH | HBTU | DIPEA | 10 | 76 |
| 8 | Fmoc-Ile-OH | HBTU | DIPEA | 10 | 80 |
| 9 | Fmoc-Lys (Boc)-OH | HBTU | DIPEA | 8 | 73 |
| 10 | Fmoc-Lys(Ddiv)-OH | HBTU | DIPEA | 8 | 75 |
| 11 | Fmoc-Lys(Fmoc)-OH | HBTU | DIPEA | 8 | 83 |
| 12 | Fmoc-Leu-OH | HBTU | DIPEA | 9 | 65 |
| 13 | Fmoc-Met-OH | HBTU | DIPEA | 7 | 73 |
| 14 | Fmoc-Asn (Trt)-OH | HBTU | DIPEA | 6 | 69 |
| 15 | Fmoc-Pro-OH | HBTU | DIPEA | 9 | 65 |
| 16 | Fmoc-Gln (Trt)-OH | HBTU | DIPEA | 10 | 71 |
| 17 | Fmoc-Arg (Pbf)-OH | HBTU | DIPEA | 10 | 70 |
| 18 | Fmoc-Ser(tBu)-OH | HBTU | DIPEA | 10 | 69 |
| 19 | Fmoc-Val-OH | HBTU | DIPEA | 8 | 66 |
| 20 | Fmoc-Trp(Boc)-OH | HBTU | DIPEA | 9 | 75 |
| 21 | Fmoc-Tyr(tBu)-OH | HBTU | DIPEA | 7 | 80 |
| 22 | Fmoc-β Ala-OH | HBTU | DIPEA | 8 | 82 |
| 23 | Boc-β Ala-OH | HBTU | DIPEA | 8 | 69 |
| 24 | Boc-Ala-OH | HBTU | DIPEA | 8 | 72 |
| 25 | Boc-Phe-OH | HBTU | DIPEA | 8 | 75 |
| 26 | Boc-Gly-OH | HBTU | DIPEA | 8 | 79 |
| 27 | Boc-Leu-OH | HBTU | DIPEA | 8 | 70 |

### Step (b): Anchoring of PG-Amino Acid-Dbz-OH (the Compound of Formula (Ib)) on the Resin

As shown in FIG. 3, the compound of formula (IIb) was obtained by anchoring PG-amino acid Dbz-OH (the compound of formula (Ib)) (step a) moiety to the solid support. A wide range of solid supports such as Rink Amide resin, chlorotrityl chloride (CTC) resin, Wang resin, Merrifield resin, PEG amine resin and Tentagel resin were applicable for this method. Based on the resin selection, the conditions for anchoring of Dbz moiety was listed in table 2 to obtain the compound of formula (IIb).

TABLE 2
The conditions for the compound of
formula (Ib) anchoring on resin

| | | | Reaction |
|---|---|---|---|
| Entry | Resin | Method | time (h) |
| 1 | Rink | Step 1: Fmoc deprotection, | 5 |
| | Amide | Step 2: Compound Ib (3.5 eq.), HBTU | |
| | | (3.5 eq.), DIPEA (8 eq.) in DMF | |
| 2 | Tentagel | Compound Ib (3.5 eq.), HBTU (3.5 | 4 |
| | | eq.), DIPEA (8 eq.) in DMF | |
| 3 | Chlorotrityl | Compound Ib (2 eq.), DIPEA (8 eq.) | 24 |
| | chloride | in DCM | |
| 4 | Wang | Compound Ib (3.5 eq.), HBTU (3.5 | 6 |
| | | eq.), DIPEA (8 eq.) in DMF | |
| 5 | Merrifield | Compound Ib (3.5 eq.), HBTU (3.5 | 5 |
| | (pre-loaded) | eq.), DIPEA (8 eq.) in DMF | |

Note:
Resins loadings were selected.

Step (c): Alternative Approach to Prepare the Compound of Formula (IIb) from Amino Acid Coupling.

As shown in the step (c) on FIG. 1, the compound of formula (IIb) was constructed with the solid phase strategy to couple PG-amino acid-OH on the compound of formula (IIa) followed a common solid phase peptide synthesis strategy. The preparation of the compound of formula (IIa) was reported on Anand Selvaraj et al (Chem. Sci. 2018, 9, 345-349). The content of Anand Selvaraj et al was incorporated into this application.

Step (d-1): Dendron Construction on the Dbz Coupled Resin

As shown in the step (d-1) on FIG. 1, the dendritic moiety was constructed through the solid phase strategy by iterative de-protection, then coupling of lysine amino acid, 3-(bis(3-aminopropyl)amino)propanoic acid, or other branch compounds based on generation number to get dendritic core on solid support (the compound of formula (IIIa)).

For example, synthetic procedure of generation-1 (G₁) lysine core was shown in FIG. 4. PG-amino acid-Dbz-OH anchored resin (step (b)) was used solid-phase Fmoc strategy for lysine core synthesis. After removal of Fmoc (10 min, 2-times, 6 mL) and resin washings (1 min, 3-times, 6 mL), the mixed solution of Fmoc-Lys(Fmoc)-OH (3.5 eq.) and HBTU (3.5 eq.) in activation solution (8 mL) was transferred to reaction vessel at room temperature for 1 h. The product was not isolated and named Go-Fmoc. After Fmoc deprotection, Boc-Lys(Boc)-OH (7 eq.) and HBTU (7 eq.) were added and stirred for 2-hour coupling to obtain G₁ lysine core with Boc groups (named G₁-Boc).

For example, the synthetic procedure of generation-2 (G₂) lysine core was shown in FIG. 5. The same procedures in FIG. 4 except for using Fmoc-Lys(Fmoc)-OH instead of Boc-Lys(Boc)-OH, that would afford G₁-Fmoc. After Fmoc deprotection, Boc-Lys(Boc)-OH (14 eq.) and HBTU (14 eq.) were added and reacted for 3 h, then, same coupling condition was repeated again (double coupling) to obtain G₂-Boc.

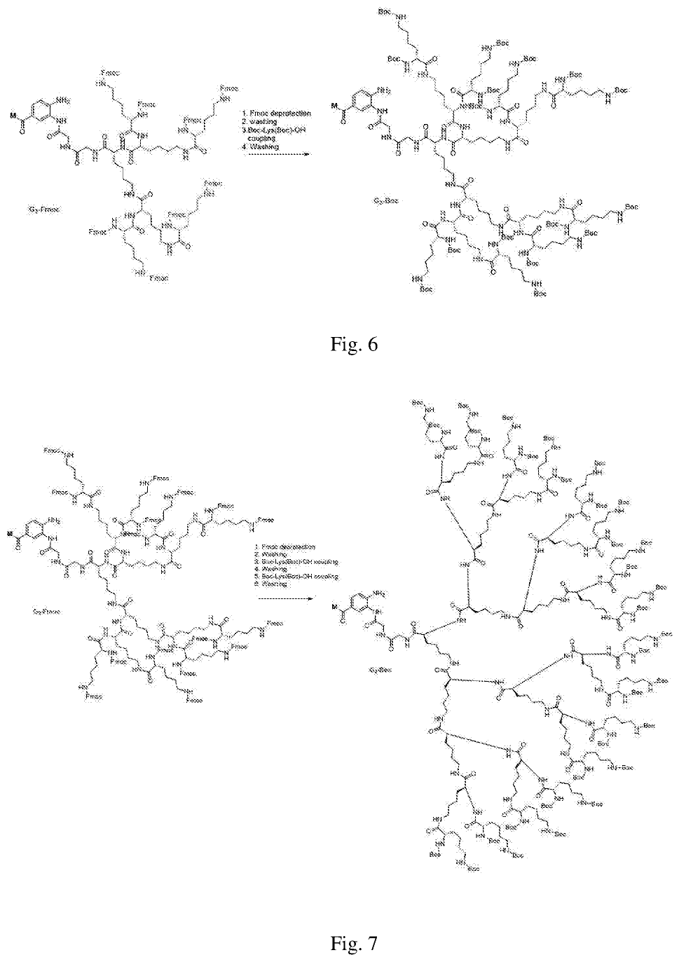

For example, synthetic procedure of generation-3 (G₃) lysine core was shown in FIG. 6. Followed same procedures recorded in FIG. 4 and FIG. 5, except for using Fmoc-Lys(Fmoc)-OH instead of Boc-Lys(Boc)-OH, it would afford G₂-Fmoc. After Fmoc deprotection, Boc-Lys(Boc)-OH (28 eq.) and HBTU (28 eq.) were added and reacted for 6 h, then same coupling condition was repeated again (double coupling) to obtain G₃-Boc.

Dendrons or dendrimers on the Dbz coupled resin would be synthesized by the above protocols. Dendron surface (the terminal groups of dendrimers or dendrons) was modified with the different amino acids as reagents instead of lysine, which was listed in the table 3. Double coupling was necessary.

TABLE 3
The conditions for synthesis of the terminal
groups of dendrimers or dendrons

| Entry | Reagents | Coupling conditions |
|---|---|---|
| 1 | Boc-Phe-OH (28 eq.)/ | Double coupling |
| | HBTU (28 eq.) | 6 h of stirring for each coupling |
| 2 | Boc-His(Boc)-OH (28 eq.)/ | Double coupling |
| | HBTU (28 eq.) | 6 h of stirring for each coupling |

N-protected amino acids other than phenylalanine and histidine could be used by following the same conditions. In addition, linear peptides, cyclic peptides, branched peptides, alkyl acids, aromatic acids, sugars, fluorescence molecules, imaging agents, targeting molecules, therapeutic agents, or peptidomimetic molecules could be conjugated on the surface. The N-protecting group is Boc, Fmoc, or others.

For example, generation-4 (G₄) lysine core synthesis procedure was shown in FIG. 7. The same procedures in FIGS. 4, 5, and 6, except for using Fmoc-Lys(Fmoc)-OH instead of Boc-Lys(Boc)-OH, afforded G₃-Fmoc. After Fmoc deprotection, Boc-Lys(Boc)-OH (58 eq.) and HBTU (58 eq.) were added and reacted for 24 h, then, same coupling condition was repeated again (double coupling) to obtain G₄-Boc.

Step (d-2): Surface of Hybrid Dendron Construction on the Dbz Coupled Resin

As shown in the step (d-2) on FIG. 1, surface hybrid dendron construction was achieved by using orthogonal protecting branched diamino carboxylate. By selectively removal of protecting group, one amine was used for preparing half part of the dendron and the other part of dendron were constructed by another amine. Then, specified surface modification was achieved. As an example, the Fmoc group of Fmoc-Lys (ivDde)-OH would be selectively cleaved for the preparing half dendron, and the ivDde protecting group would be removed with hydrazin solution for the preparation of other dendrons.

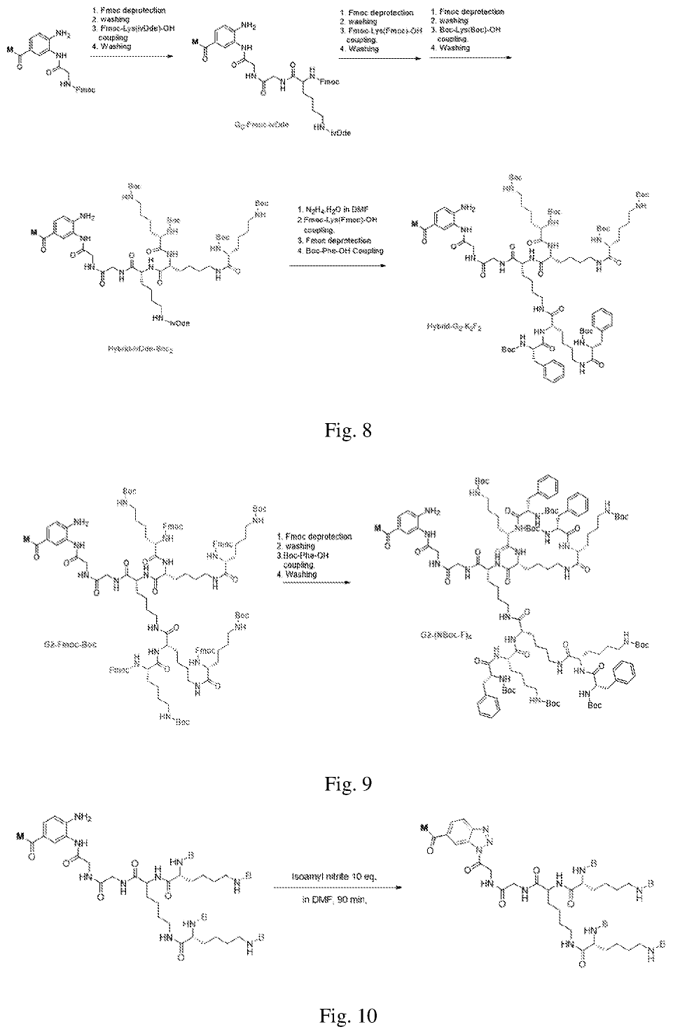

For example, the construction of hybrid dendron was showed in FIG. 8. Modification of Fmoc-Lys-Dbz-OH anchored resin was followed by Fmoc deprotection and Fmoc-Lys(ivDde)-OH coupling to obtain Go-Fmoc-ivDde. After Fmoc removal, typical solid-phase peptide synthesis was proceeded by consecutive Fmoc-Lys(Fmoc)-OH coupling, Fmoc deprotection, and Boc-Lys(Boc)-OH coupling to generate half dendron to offer compound named Hybrid-ivDde-Boc₂. Thereafter, another half dendron terminated with phenylalanine was created after removal of ivDde, and consecutive Fmoc-Lys(Fmoc)-OH coupling, Fmoc deprotection, and Boc-Phe-OH coupling to obtain Hybrid-G₂-K₂F₂.

For example, amino acid-modified hybrid dendron was shown in FIG. 9. As the similar procedure described in FIG. 4, Go-Fmoc was prepared. Later, G₁ product would be synthesized after Go-Fmoc by using Fmoc-Lys(Fmoc)-OH instead of Boc-Lys(Boc)-OH to afford G₁-Fmoc but not G₁-Boc. Followed similar conditions in FIG. 5 to synthesize G₂ product, Fmoc-Lys(Boc)-OH was used instead of Boc-Lys(Boc)-OH to obtain G₂-Fmoc-Boc but not G₂-Boc. After deprotection of Fmoc, Boc-Phe-OH coupling was subjected to generate a hybrid dendron terminated with phenylalanine, named G₂-(NBoc-F)₄.

### Step (e): General Procedure for On-Bead Activation of Dbz Moiety to Acylated Benzotriazole:

As shown in the step (e) of FIG. 1, the resin-bound Dbz dendron was washed with DMF (5-10 mL) and DCM (5-10 mL), then transferred into glass vial. Resin was treated with isoamyl nitrite (10 eq.) in DMF (5-8 mL) for 90 min at room temperature to generate on-bead acylated benzotriazole. The remaining reagents was washed out with DMF (5-10 mL) and DCM (5-10 mL).

For example, on-bead activation for different generation compounds showed at FIG. 10. The resin-bound Dbz peptide-dendron was washed with DMF (3 mL×3) and DCM (3 mL×3), then transferred into 20 mL glass vial. Resin was treated with isoamyl nitrite (10 eq.) in DMF (4 mL) and shaken for 90 min at room temperature. The remaining reagents was washed with DMF (3 mL×3) and DCM (3 mL×3). In FIG. 10, B in the compounds was selected from:

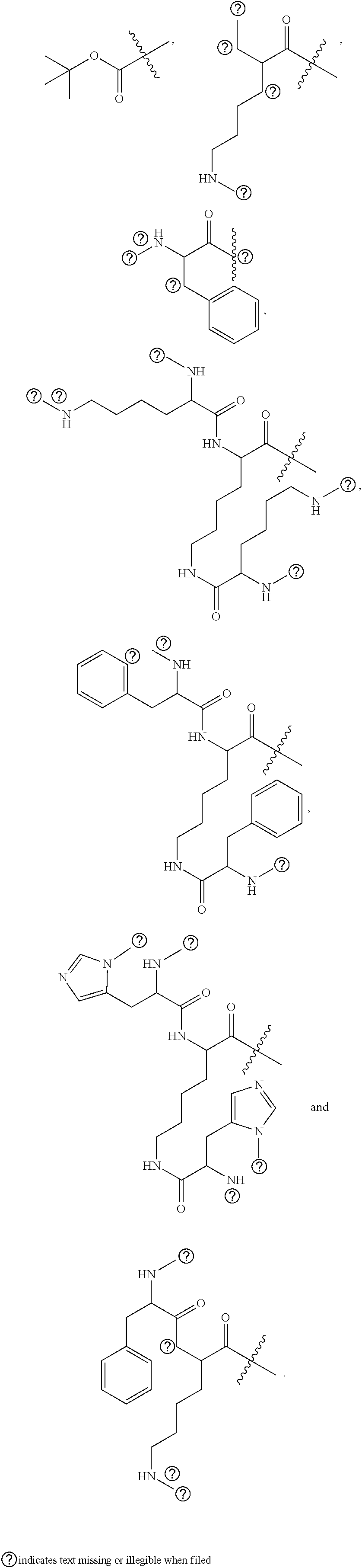

### Step (f): General Procedure for on Resin Nucleophilic Substitution

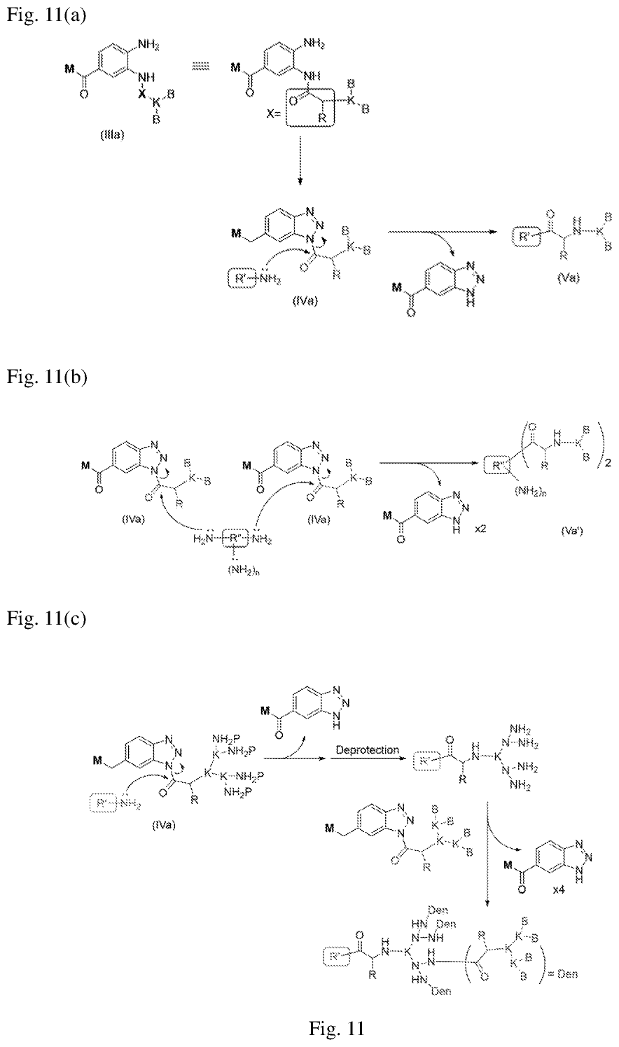

After treated with isoamyl nitrite, the resin-bound Dbz peptide-dendrons (the compound of formula (IIIa) or (IIIb)) had been activated to form compounds with bezotriazole moiety (the compound of formula (IVa) or (IVb)). In the step (f-1) and (f-2) of FIG. 1, compound with monoamine was used to attack the compound of formula (Iva) or (IVb), and deattached the compound of formula (Va) or (Vb) was obtained. Alternatively, multi-amine was used to attack the compound of formula (IVa) or (IVb) twice, and deattached the compound of formula (Va′) or (Vb′). The detail mechanisms were presented in FIG. 11. As shown in FIG. 11(a), the monoamine attacked the carbonyl group connected on triazole ring of the compound of formula (IVa) and substituted the benzotriazole moiety to detach the compound of formula (Va) from solid support. The preparation of the compound of formula (Vb) was followed similar protocol to attack the compound of formula (IVb). In FIG. 11(b), a di- or multi-amine was the nucleophile. Each amino group of di- or multi-amine would all attacked the carbonyl groups on the compound of formula (IVa) to generate two or more substitutions (the compound of formula (Va′)). The preparation of the compound of formula (Vb′) was followed similar protocol to attack the compound of formula (IVb). High yields were observed with microwave irradiation. Nucleophilic substitution was performed with the different cocktail combinations based on the solubility of each nucleophile. For example, substitution with nucleophiles having pyrene or C18 derivatives was done in combination of DCM and DMF. Substitution with nucleophiles having adamantyl moiety or hydrophobic cyclic peptide was done in DMF. Alternatively, substitution with polar organic molecules was done in DMSO and DMF combination. Followed the similar protocol, di- or multi-amine could be possible to attack the compounds of formula (IVa) and (IVb) to obtain a hybrid product.

As shown FIG. 11(c), in the case of the synthesis of higher generation dendrimers, the amphiphilic dendron itself could act as a nucleophile bearing multiple amines to produce higher generation of dendrons in a more efficient manner, which did not synthesize target dendron from generation to generation stepwise (step (f-2)).

On-resin Dbz activated resin was transferred into microwave vial (8 mL) with DMF. DIPEA (8 eq.) was added and stirred for 5 min. Then, the solution of nucleophile (1 eq.) in solvent was added. Microwave reaction was carried out at 50° C., 100 W for 10 min. High yields and purity was observed.

Different nucleophiles along with the different cocktail combination was listed in the below table 4.

TABLE 4
Different nucleophiles and different cocktail combination thereof

| Entry | Nucleophile | Type of amine | Cocktail |
|---|---|---|---|
| 1 | Stearylamine | Mono-amine | DMF:DCM (3:1) |
| 2 | Amine with Double | Mono- amine | DMF:DCM (2:1) |
| | alkyl chain | | |
| 3 | Pyrene amine | Mono- amine | DMF:DCM (2:2) |
| 4 | Adamantyl amine | Mono- amine | DMF |
| 5 | Hydrophobic cyclic | Mono- amine | DMF |
| | peptide | | |
| 6 | Dopamine amine | Mono- amine | DMSO:DMF (1:3) |
| 7 | G₀ amphiphilic | Multi-amine | DMF:DCM (3:2) |
| | dendrimer | | |

Note:
Reaction was done at 50° C., 100 W for 10 min, 17 psi.

### General Procedure for Final De-Protection and Purification

Surface protection groups were de-protected with selective cocktail combinations. For example, Boc group was cleaved with TFA cocktail and Fmoc group was cleaved with piperdine or diethylamine (DEA) in DMF.

Once the nucleophilic Substitution was done, protected crude compound was collected after filtrated resin out, and the solution was concentrated. Cleavage solution (TFA: H₂O/95:5) (2-7 mL) was added and the reaction mixture was shaken (200 rpm) at room temperature for 1-4 h to remove the Boc group. Cleavage solution (DEA: DCM/1:1) (4-8 mL) was added and the reaction mixture was shaken (200 rpm) at room temperature for 3-8 h to remove the Fmoc group.

After protection removal, the amphiphilic products can be collected by common precipitation to obtain compounds with high purities. The lysine based dendrimer with different hydrophobic nucleophiles were precipitated out in cold ether and centrifugation was followed with above 5500 rpm for 5 min. Precipitated compound was separated by decant. The resulting compound was dissolved in 2-5 mL of the mixed solvents of water and ACN (1:1) containing 0.1% TFA and allowed for lyophilization to get target products.

If amphiphilic compound contained 3-(bis(3-aminopropyl)amino)propanoic acid based dendron, it needed to be purified by column chromatography with 3-8% of methanol in DCM to get the target compounds.

Those skilled in the art recognize the foregoing outline as a description of the method for communicating hosted application information. The skilled artisan will recognize that these are illustrative only and that many equivalents are possible.

What is claimed is:

1. A method for synthesizing amphiphilic dendrimers, comprising: (a) reacting a compound of formula (IIb) with at least one diamino carboxylate having two protecting group for replacing Y on the compound of formula (IIb) to develop branched skeletons and modifying the terminal groups of the branched skeleton with functional moieties to obtain a compound of formula (IIIb), 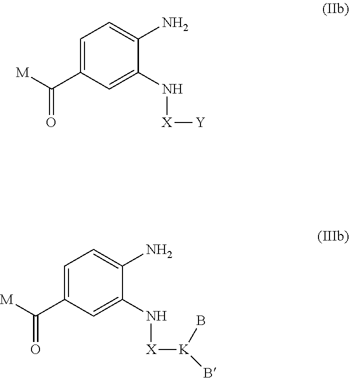
wherein M is a solid support material;
X is amino carboxylate;
Y is a protection group to protect the N-terminal of X;
K is a moiety with branched diamine;
B and B′ are K₍₂_{ⁿ}₋₁₎Z₂_{ⁿ}, wherein n is 0 to 5 and Z is a functional moiety comprising H, amino acids, peptides, acids, sugars, targeting ligands, imaging molecules, therapeutic agents, or peptidomimetic molecules;
provided that B is not the same as B′, at least one of Z, K or n of B is different from B′;
(b) reacting the compound of formula (IIIb) with nitrite in a polar solvent to obtain a compound of formula (IVb) having a benzotriazole structure, 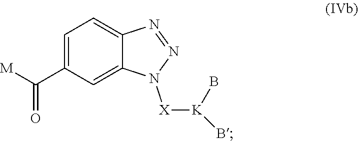
and
(c) reacting the compound of formula (IVb) with one amine having hydrophobic residues in a mixed solution containing a hydrophilic solvent and a hydrophobic solvent to obtain a compound of formula (Vb), 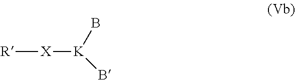
wherein R′ comprises amino groups having hydrophobic residues; or reacting at least two compounds of formula (IVb) with corresponding di- or multi-amine having hydrophobic residues in a mixed solution containing a hydrophilic solvent and a hydrophobic solvent to obtain a compound of formula (Vb′), 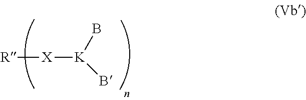
wherein R″ comprises branched di- or multi-amino amino groups having hydrophobic residues, and n>1.

2. The method of claim 1, which further comprises step (pre-a2), before step (a), comprising immobilizing a compound of formula (Ib) on a solid support material to obtain the compound of formula (IIb),

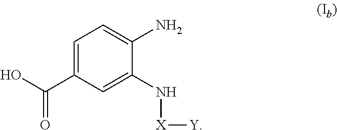

3. The method of claim 2, which further comprises step (pre-a1), before step (pre-a2), comprising reacting a compound of formula (Ia) with a compound having the X-Y to obtain the compound of formula (Ib),

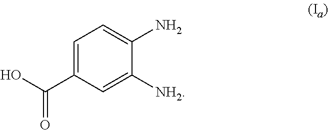 [SMILES: Nc1ccc(C(=O)O)cc1N]

4. The method of claim 1, wherein the solid support material comprises controlled-pore glass, magnetic beads, Rink amide resin, Tentagel resin, Wang resin, Merrifield resin, MBHA resin, PAM resin, PAL resin, Sieber Amide resin, trityl resin, chlorotrityl resin, Polyethylene Glycol-Polystyrene resin, Weinreb resin, oxime resin, DHP resin or Safety-catch resin.

5. The method of claim 1, wherein the X comprises: 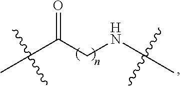
wherein n is an integer, from 1 to 10; or 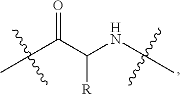
wherein R is H, —CH₃, —CH(CH₃)₂, —CH₂CH(CH₃)₂, —CH(CH₃)CH₂CH₃, —CH₂C₆H₅; the O-PG derivatives of —CH₂COOH, —CH₂CH₂COOH, —CH₂OH, —CH₂CH(CH₃)OH or —CH₂C₆H₄OH; the S-PG derivatives of CH₂SH or —CH₂CH₂SCH₃; the N-PG derivatives of —CH₂CH₂CONH₂, —CH₂CONH₂, —CH₂CH₂CH₂CH₂NH₂ or —CH₂CH₂CH₂NHC(NH)NH₂; or
the N-PG derivatives of 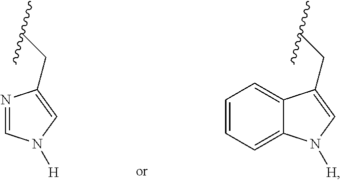
wherein PG is a protecting group to protect N, O, or S.

6. The method of claim 1, wherein the Y comprises 9-fluorenylmethoxycarbonyl, tert-Butoxycarbonyl, Benzyl carbamates, Benzy or Alloxycarbonyl groups.

7. The method of claim 1, wherein the form of the B or B′ comprises the branched skeletons comprising KZ₂, K(KZ₂)₂, K(K(KZ₂)₂)₂, K(K(K(KZ₂)₂)₂)₂, or K(K(K(K(KZ₂)₂)₂)₂)₂.

8. The method of claim 1, wherein the R′ comprises 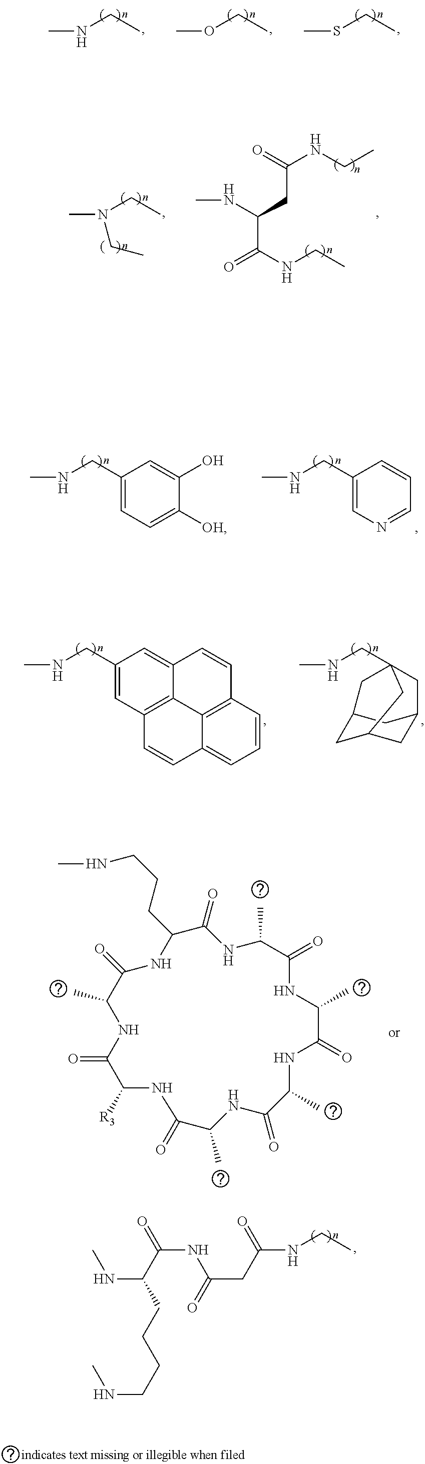
wherein n is 0 to 20, and R₃ is a side chain of amino acids.

9. The method of claim 1, wherein the nitrite comprises sodium nitrite, potassium nitrite, ethyl nitrite, butyl nitrite, t-butyl nitrite, isobutyl nitrite, pentyl nitrite, isopentyl nitrite, iso-amyl nitrite, dicyclohexylamine nitrite or tetrabutylammonium nitrite.

10. The method of claim 1, wherein the polar solvent in the step (b) comprises dichloromethane, N,N-dimethylacetamide, N,N-dimethylformamide, N-methyl-pyrrolidone, dichloroethane, trichloroethane or water.

11. The method of claim 1, wherein the hydrophilic solvent in the step (c) comprises dimethyl sulfoxide, N,N-dimethylformamide, N,N-dimethylacetamide, N-methyl-pyrrolidone or N-butylpyrrolidinone.

12. The method of claim 1, wherein the hydrophobic solvent in the step (c) comprises dichloromethane, dichloroethane, dichlorobenzene, 1,1,2-trichloroethane, tetrahydrofurane, dioxane or ethyl acetate.

13. The method of claim 1, wherein the reacting temperature of the mixed solution ranges from 1 to 150° C.

14. The method of claim 1, wherein the volume ratio of the hydrophilic solvent and the hydrophobic solvent ranges from 50:0 to 1:15.

15. The method of claim 1, which further comprises a step (d), after the step (c), comprising removing the protecting groups of the compound of formula (Vb) or (Vb′).
